(19) United States
(12) Patent Application Publication
(10) Pub. No.: US 2009/0326334 A1
(43) Pub. Date: Dec. 31, 2009

(54) HEALTH CONDITION DETECTION, STORAGE, AND PRESENTATION

(76) Inventor: Anthony Vallone, North Canton, OH (US)

Correspondence Address: ESCHWEILER & ASSOCIATES, LLC;NATIONAL CITY BANK BUILDING, 629 EUCLID AVE., SUITE 1000, CLEVELAND, OH, 44114, US

(21) Appl. No.: 12/147,383

(22) Filed: Jun. 26, 2008

(51) Int. Cl. A61B 5/00; G06Q 50/00

(52) U.S. Cl. 600/300; 705/3

(57) ABSTRACT

Health-related devices often record and represent health-related information describing a patient in many manners, and the disparities and variance among such representations may interfere with the accuracy of a recorded electronic medical record and with communication among healthcare providers. These aspects of health information representation and communication may be facilitated according to several techniques, such as by storing health information according to a standardized health information hierarchical classification scheme, and by presenting such information to healthcare providers and the user by displaying one or more icons of predominantly pictorial form that describe the health information in a language- and literacy-independent manner. Variations of these techniques relate to the types of devices so presented, the storage of the detected health data with the representation, and the annotation of the data by the individual or healthcare providers that becomes part of the electronic medical record.

### BACKGROUND

Many scenarios within the broad context of healthcare (including, e.g., inpatient, outpatient, home health caregivers, and assisted-living healthcare settings) involve the recording of a measurement, such as blood pressure, heart or respiratory rate, visual measurement of an anatomic feature such as a wound, and temperature measurement. In some instances, these measurements are detected and output by a healthcare device, such as a blood pressure cuff, which healthcare providers of the individual may monitor for information utilized in the healthcare needs of the individual. In other instances, these measurements may be recorded on a storage device, which healthcare providers may access and analyze. However, the information yielded by such devices is not generally stored as part of the individual's medical record, and where such information may be so incorporated, it is typically included as raw data with a healthcare provider's annotations.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

Many types of data may be generated by various healthcare devices (such as sensors) in relation to the healthcare needs of an individual, and may be utilized in various ways in furtherance of the healthcare interests of the individual. This data may be advantageously integrated into the individual's electronic medical record. Moreover, the integration may exhibit various qualities. First, the integration may be based on a health information hierarchical classification scheme, which may be broadly devised to include descriptors for various health conditions that may apply to the individual. The health information hierarchical classification scheme may include a set of health condition descriptors and a related set of health condition ratings that may be specified together to characterize aspects of the individual's health state in categorical ways, which may be helpful for evidence-based healthcare evaluation. Moreover, the information generated by such healthcare devices may be represented and stored according to the health information hierarchical classification scheme. Second, the various descriptors of the health information hierarchical classification scheme may be associated with a standardized set of icons of predominantly pictorial form, such that similar concepts may be consistently illustrated in an easily understood manner, even for viewers with differing language and literacy capabilities. By tracking a wide assortment of healthcare-related data in a standardized health information hierarchical classification scheme and displaying the data through icons of predominantly pictorial form, the computer systems involved in the healthcare of the individual may be more advantageously structured to facilitate healthcare providers in the care of the individual.

To the accomplishment of the foregoing and related ends, the following description and annexed drawings set forth certain illustrative aspects and implementations. These are indicative of but a few of the various ways in which one or more aspects may be employed. Other aspects, advantages, and novel features of the disclosure will become apparent from the following detailed description when considered in conjunction with the annexed drawings.

### DESCRIPTION OF THE DRAWINGS

FIG. 1 is an exemplary scenario illustrating techniques for the detection, representation, and presentation of health-related information of an individual.

FIG. 2 is a flow chart illustrating an exemplary computer-implemented method of representing a health condition of an individual.

FIG. 3 is a component block diagram illustrating an exemplary system for representing a health condition of an individual.

FIG. 4 is an illustration of a detecting of a measurement of a health condition at different times and representing of the measurement according to a health information hierarchical classification system.

FIG. 5 is an illustration of an exemplary computer-readable medium comprising processor-executable instructions configured to embody one or more of the provisions set forth herein.

FIG. 6 illustrates an exemplary computing environment wherein one or more of the provisions set forth herein may be implemented.

### DETAILED DESCRIPTION

The claimed subject matter is now described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

In the broad set of fields that generally relate to the healthcare of an individual (including, e.g., inpatient and outpatient hospital, medical office, home healthcare, and assisted-living settings), a wide array of devices are commonly used to measure many types of data relating to the health state of the individual. Such measurements typically relate to a particular health condition (e.g., cardiovascular health, respiratory health, mental cognition, and ocular health) and may comprise a wide assortment of data (e.g., blood pressure, respiratory rate, a brain scan illustrating mental activity, and intraocular pressure.)

The tracking of these myriad forms of healthcare-related data is often disjointed. For example, some forms of data may be observed by a healthcare provider and used while providing for the healthcare needs of the individual, and may be recorded in the individual's medical record (electronic or hard copy) only as a matter of course. Other forms of data may be electronically stored by the device, such as a medical image stored on a medical imaging server; such data may be stored in a data archive for future access, but may only loosely be incorporated in the medical record (e.g., by reference to the existence of such data.) Still other forms of data may be integrated with the medical record as a data set (e.g., a medical image stored as a bitmap in the electronic medical record, or a set of readings from a heart monitor), but may not be electronically integrated with the semantic representation of the health state of the individual; i.e., the data may not be representationally linked to any particular aspect of the health state of the individual, nor to other readings that may be indicative of the condition thereof. Moreover, these forms of data may be stored in different ways by different healthcare providers. For instance, a first healthcare provider may document a blood test in the medical record according to a set of numeric measurements by a first healthcare provider, such as “hematocrit: 46%”, as reported directly by a chemical detection device. However, a second healthcare provider may record the same information as a cursory description by a second healthcare provider, such as “blood test: negative,” while a third healthcare provider may incorporate the information as part of a healthcare narrative, such as “the individual was subjected to blood tests that did not provide any helpful information.”) The variance by different healthcare providers in representing health information, and particularly in healthcare-related data generated by various types of detectors, may complicate an automated evaluation of the health state of the individual. This may also interfere with communication among healthcare providers, particularly where the languages or literacy capabilities of such healthcare providers may vary (e.g., a physician may create a medical record in the French language that may not be cognizable to a healthcare aide with a poor understanding of either French or medical jargon.)

Alternative techniques for representing and presenting data generated by healthcare-related devices may be devised that improve the standardization of representation among healthcare providers, and that promote communication of such information thereamong. One such technique involves representing the information in a standardized format, such as may be defined by a health information hierarchical classification scheme, which may define different sets of related categories for describing different aspects of an individual's health state. The relational structure of the classification scheme may be embodied, for example, in a relational database, and information generated by many healthcare providers (even of differing languages and/or literacy levels) may be more uniformly stored and represented, thereby improving the standardization of the electronic medical record of the individual. One such health information scheme is disclosed in U.S. patent application Ser. No. 11/753,306 (“Health Information Hierarchical Classification Scheme and Methods and Systems Related Thereto”), the entirety of which (except the claims) is incorporated herein by reference. Another such health information scheme is the Outcomes and Assessments Information Set (OASIS) classification scheme created and utilized by the U.S. Department of Health & Human Services.

In one such health information hierarchical classification scheme, the health information hierarchical classification scheme comprises a set of health conditions that describe different aspects of the individual, such as cardiovascular health and respiratory health. A health condition may be related to a set of health condition descriptors that describe various aspects of the health condition, and a set of health condition ratings that describe qualities and different levels of wellness for different health condition descriptors. For example, a health condition representing cardiovascular health may include health condition descriptors such as heart rate, blood pressure, and coagulation rate, and health condition ratings such as dangerously high, normal, low, and dangerously low. The cardiovascular health of an individual may therefore be described by selecting one or more combinations of a health condition descriptor and a health condition rating, which may be stored together, e.g., in a relational database configured according to the health information hierarchical classification scheme.

Another technique for improving the representation and presentation of information in an electronic medical record relates to the communication of health information in a pictographic form that is easy for the individual to recognize and comprehend. The use of an icon set for communicating health-related information has many potential advantages. First, the individual and healthcare providers may comprehend the information in a language-independent manner. Pictographic icons can be used to circumvent differences in language capabilities and skill levels among the caregivers and the individual. The icon set may be cognizable to individuals with limited literacy skills or with mental faculties, such as very young, elderly, and mentally handicapped individuals. Second, if a common set of health information icons is utilized by many caregivers and individuals, it may become a standard and standardized lexicon for the communication of health-related information, including medication regimen information. Regular and frequent exposure to the same icon set, especially from a variety of sources, may lead to quicker, fuller, and more accurate recognition and comprehension by caregivers and individuals of these icons and the illustrated concepts. Moreover, widespread exposure to and use of the same icon set may improve the speed, depth, and precision of communication of health-related information among many caregivers and individuals.

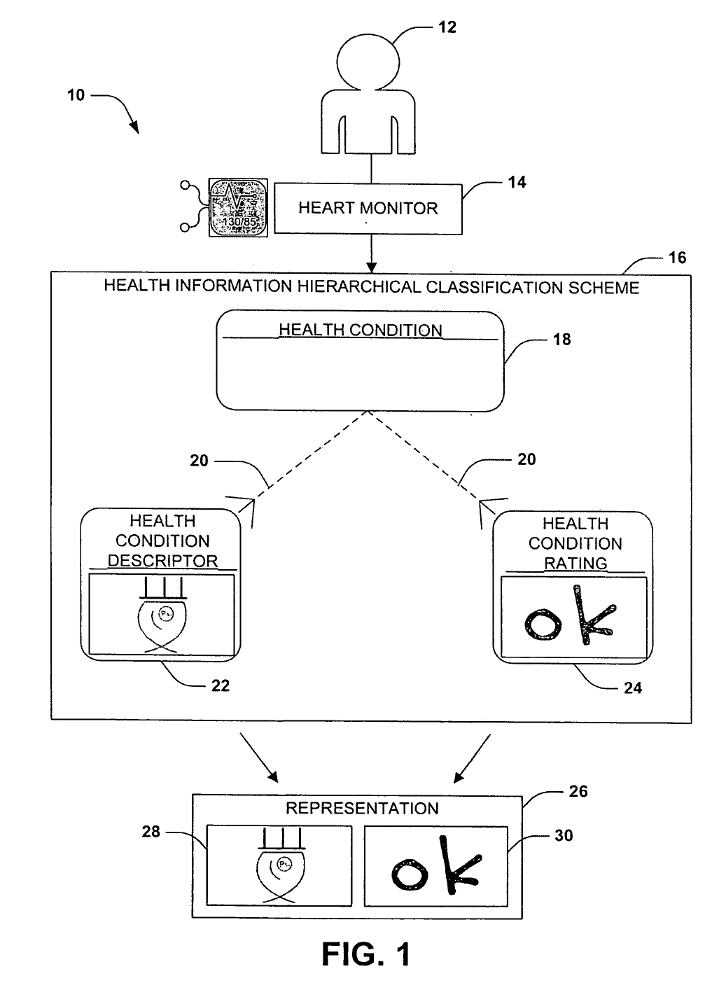

FIG. 1 presents a scenario 10 that illustrates the combination of these techniques in order to represent and present health information of an individual in an advantageous manner. The scenario 10 involves an individual 12 who is subjected to a heart monitor 14 that detects and records information about the cardiovascular health of the individual 12, including the blood pressure of the individual 12. According to these techniques, the recorded information is represented according to a health information hierarchical classification scheme 16, which is illustrated in FIG. 1 in Unified Modeling Language (UML) representation. This health information hierarchical classification scheme 16 includes a set of health conditions 18, each of which has relational links 20 with one or more health condition descriptors 22 and one or more health condition ratings 24. In this scenario 10, the blood pressure information recorded by the heart monitor 14 is represented according to a health condition 20 representing cardiovascular health, and more specifically by a selected health condition descriptor 22 representing “blood pressure” and a selected health condition rating 24 representing a “normal” rating. The selected descriptors together form a representation 26 of the measured aspect of the health state of the individual. Moreover, the representation 26 may be conveyed among healthcare providers and the individual through the display of icons of predominantly pictorial form that are associated with the descriptors of the health information hierarchical classification scheme 20. Thus, the representation 26 may be presented by displaying a first icon 28 depicting the concept of blood pressure as represented by the selected health condition descriptor 22, and a second icon 30 depicting the concept of a “normal” rating as represented by the selected health condition rating 24. By representing and conveying the health-related information of the individual 12 in this manner, the scenario 10 illustrates an advantageous configuration of the electronic medical record of the individual 12 that improves the consistency of stored health information and the communication of health information among various users.

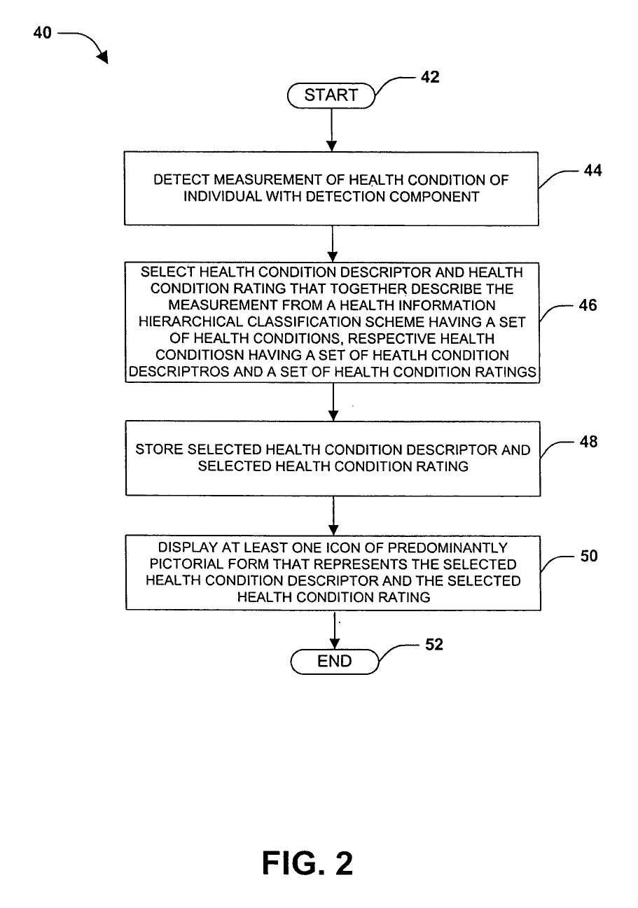

FIG. 2 illustrates a first embodiment of this technique, comprising an exemplary computer-implemented method 40 of representing a health condition of an individual. The exemplary computer-implemented method 40 begins at 42 and involves detecting 44 a measurement of the health condition of the individual with a detection component (e.g., a heart monitor.) The exemplary computer-implemented method 40 also involves selecting 46 a health condition descriptor and a health condition rating that together describe the measurement from a health information hierarchical classification scheme having a set of health conditions, where respective health conditions have a set of health condition descriptors and a set of health condition ratings. The exemplary computer-implemented method 40 also involves storing 48 the selected health condition descriptor and the selected health condition rating. Finally, the exemplary computer-implemented method 40 also involves displaying 50 at least one icon of predominantly pictorial form that represents the selected health condition descriptor and the selected health condition rating. By translating the measured health information data into the format of the health information hierarchical classification scheme and presenting it through icons of predominantly pictorial form, the exemplary computer-implemented method 40 therefore achieves the storage and communication of the health-related information in an consistent and easily cognizable manner, and so ends at 52.

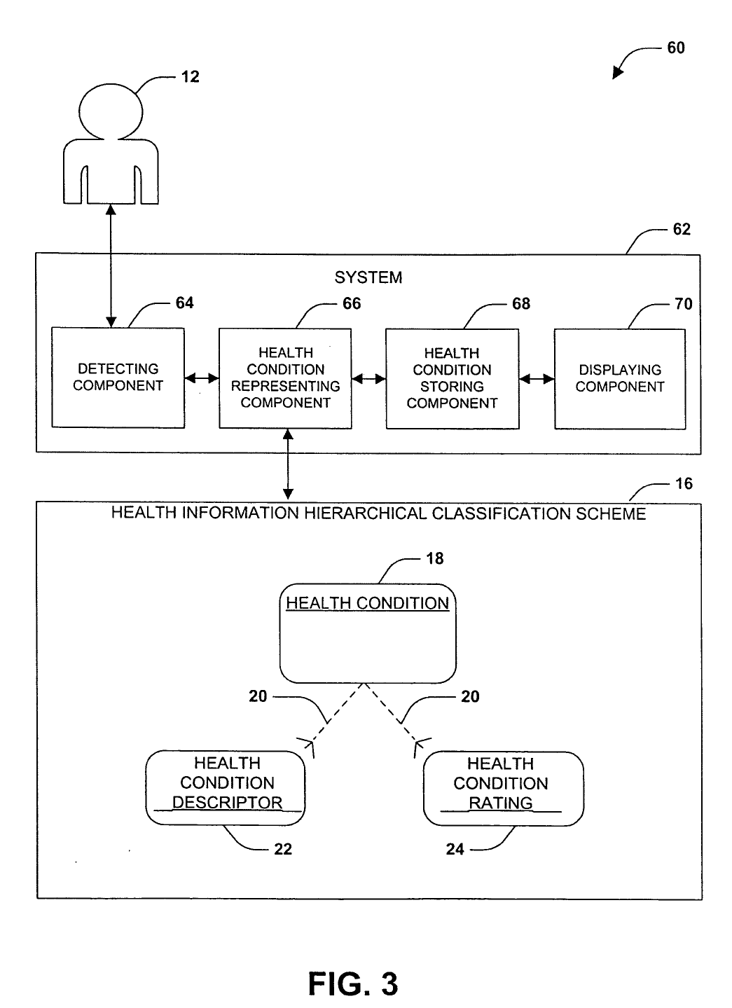

FIG. 3 illustrated a scenario 60 featuring another embodiment, comprising an exemplary system 62 configured to representing a health condition of an individual. The exemplary system 62 of FIG. 3 may be utilized to detect, represent, and present the health information of one or more individuals 12 according to the techniques discussed herein. The exemplary system 62 features a detecting component 64, which is configured to detect a measurement of the health condition of the individual that relates in some way to the health state of the individual. The exemplary system 62 also includes a health condition representing component 66, which is configured to select a health condition descriptor and a health condition rating that together describe the measurement of the health condition of the individual 12, from a health information hierarchical classification scheme 16 having a set of health conditions 18, where respective health conditions 18 have relational links 20 with a set of health condition descriptors 22 and a set of health condition ratings 24. The exemplary system 62 also includes a health condition storing component 68, which is configured to store the selected set of health conditions 18. Finally, the exemplary system 62 includes a displaying component 70, which is configured to display at least one icon of predominantly pictorial form that represents the selected health condition descriptor and the selected health condition rating. The components of the exemplary system 62 cooperate according to the techniques herein to achieve the detection, representation, and presentation of the health-related information of the at least one individual 12.

The techniques presented herein may include variations in certain aspects, and some variations may present advantages and/or reduce disadvantages with respect to other variations of these and other techniques. Moreover, some variations may be compatible with other variations, and some combinations may present synergistic advantages. Such variations may also be implemented, alone or in combination, in various embodiments (such as the exemplary method 40 of FIG. 2 and the exemplary system 62 of FIG. 3) to confer the advantages and/or mitigate disadvantages related thereto.

A first aspect that may vary among implementations of these techniques relates to the detecting of health-related information. Many types of detection and detecting components may be included in such embodiments. For example, the detecting may comprise a visual detection component, such as a camera, that is configured and positioned to capture an image relating to the health state of the individual, such as a dental X-ray film, an MRI image of an anatomic feature, or a microscopic view of a tissue sample prepared in a pathology lab. Such medical images are often analyzed to produce health-related information that may be more discretely captured as part of the individual's electronic medical record; for example, a dental X-ray film may yield health information about particular teeth (e.g., an access cavity on a lower first left molar); an MRI image may yield information about an anatomic feature of interest (e.g., a size dimension of a suspicious tumor); and a microscopic view of a tissue sample may yield information about cellular quantities, types, or structures (e.g., an approximate red blood cell count.) An audio detection component may be utilized to detect audible aspects of the health state of the individual, such as a heart murmur, an unusual respiratory sound, or sound from the abdomen indicative of the health of the digestive track. A temperature detection component may be utilized to monitor the temperature of various anatomic features, such as a fever monitor. A pressure detection component may be utilized to monitor various pressure aspects, such as blood pressure and intracranial fluid pressure. A flow detection component may be utilized monitor the flow of liquids or gases, such as blood flow, liquid drug dispensation, or catheter activity. A chemical detection component may be utilized to apply various health-related chemical tests, such as blood glucose and detection of bacteria. Many types of health-related detectors may be utilized in detecting a measurement to be represented according to the techniques discussed herein. The detecting may also be configured to receive user input, such as received from a user (e.g., the individual, or a healthcare provider for the individual) through a user input component, where the user input represents a selection of a health condition rating. A user may be facilitated in detecting and inputting such information through the use of an icon-based task performance facilitator, such as disclosed in U.S. Patent App. No. 61/018,191 (“Icon-Based Facilitation of Service Task Performance”), the entirety of which (except the claims) is incorporated herein by reference. This manner of detecting may be useful for aspects of the health state of the individual that may not be easily detected in an automated manner, such as a degree of pain experienced from an injury.

Alternatively or additionally, the user input may comprise an annotation of a measurement, such as an observation by a healthcare provider that the measurements of a wound seem to indicate that the wound is healing well in response to a particular applied medicament. In such embodiments, the detecting may involve receiving from the user a health condition annotation through the user input component; the storing may comprise storing the health condition annotation with the selected health condition descriptor and the selected health condition rating; and the displaying may comprise displaying the health condition annotation with the at least one icon of predominantly pictorial form. However, those of ordinary skill in the art may select and configure many detection components to detect and evaluate the measurement of the health condition of the individual in accordance with the techniques discussed herein.

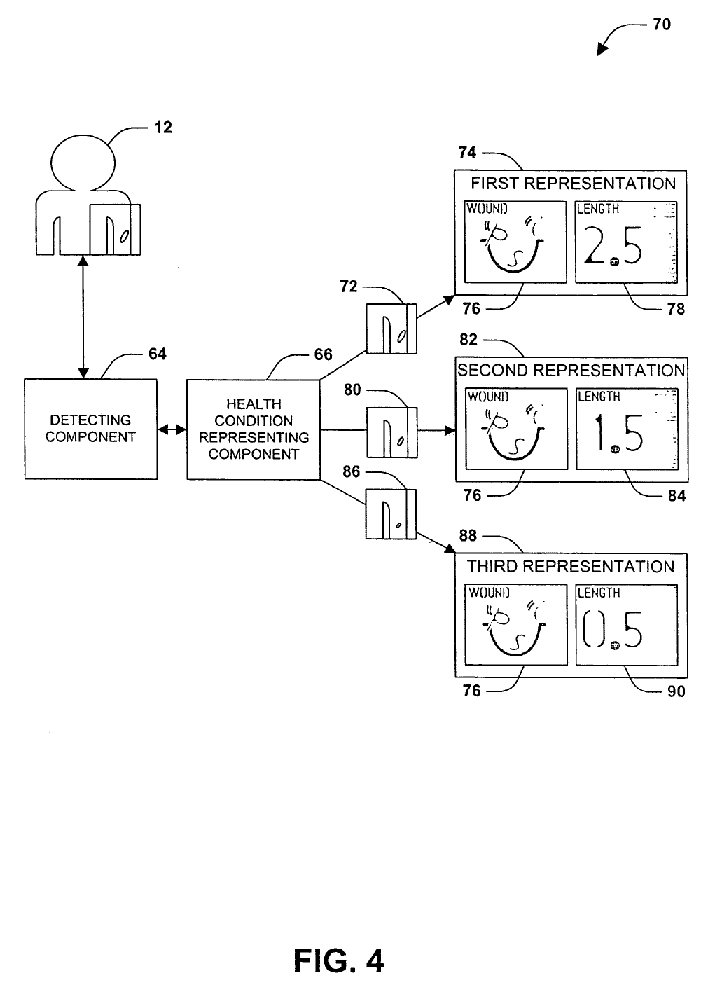

FIG. 4 illustrates a particular example 70 of detecting of health-related information involving a wound on the left arm of an individual 12. The wound may be captured with a detecting component 64 with a visual detector (e.g., a camera), and some properties of the image may be selected by a health condition representing component 66 for representation in the electronic medical record to describe the condition of the wound, such as its length, width, depth, color, and appearance (rough, smooth, scabbed, etc.) For example, a first image 72 may be captured, and a first health condition representation 74 may be generated therefrom based on the first image 72. The first representation 74 may include a health condition descriptor 76 that identifies the recorded aspect as a wound length, and from the first image 72, a first health condition rating 78 may be selected that describes the wound as “large” in length (more than two inches.) At a later time, after the wound has healed to a certain extent, a second image 80 may be recorded, and a second health condition representation 82 may be generated therefrom comprising the same health condition descriptor 76 and a second health condition rating 84 now describing the wound as of “medium” length (1.0 inches to 2.0 inches.) Still later, a third image 86 may be captured and a third health condition representation 88 generated therefrom, comprising the same selected health condition descriptor 76 and a third health condition rating 90 indicating that the wound is now “small” in length. This selecting may be performed, e.g., by an image analysis algorithm that is trained to evaluate images of wounds and generate measurements therefrom, such as dimensions and relative color. Other detectors may also be applied in relation to the evaluation of other properties of the wound, such as a thermometer applied to measure the temperature of the wound as an indication of perfusion and inflammation, and a moisture detector to determine whether the wound is seeping or dry.

A second aspect that may vary among implementations of these techniques relates to the selecting of the health condition descriptor and the health condition rating based on the detected measurement. Many types of measurements may be represented according to such selections. As a first example, the descriptors selected for the representation may comprise a quantitative measurement, such as various ranges of parameters (e.g., different quantitative size measurements of a wound, as illustrated in FIG. 4.) Alternatively or additionally, the descriptors may comprise a qualitative measurement, such as “large,” “medium,” or “small” to represent a dimension of a wound. The measurement may also be represented relative to a measurement standard (e.g., “high,” “normal,” or “low” to represent a temperature measurement as compared with a measurement standard of a normal body temperature of 98.6° C.) The measurement may also be represented relative to a prior measurement, such as a wound size that is measured as “larger,” “same,” or “smaller” as compared with a prior recorded measurement. The measurement may also be represented as relative to a predicted measurement, such as a wound dimension represented as “larger than predicted,” “as predicted,” or “smaller than predicted” relative to a predicted healing process. Many such manners of selecting the measurement may be devised by those of ordinary skill in the art while implementing the techniques discussed herein.

A third aspect that may vary among implementations of these techniques relates to the storing of the selected representation as a part of the electronic medical record. As a first variation, the selected representation may be stored locally in a file system of the detector device, and may be later retrieved for analysis. Alternatively or additionally, the selected representation may be stored in a remote server, such as an electronic medical record database configured to store health information representations for various individuals. The storing may also comprise the measurement with the selected health condition descriptor and the selected health condition rating. For example, in addition to recording the status of a photographed wound according to the descriptors of the health information hierarchical classification system (e.g., with a “wound length” of “large”), the storing may involve storing the actual measurement generated by an image analysis algorithm, and/or the actual image of the wound. Those of ordinary skill in the art may devise many ways of storing the representations of the measured and represented health condition while implementing the techniques discussed herein.

A fourth aspect that may vary among implementations relates to the manner of displaying the icons of predominantly pictorial form. The displaying is likely to be performed by displaying the at least one icon of predominantly pictorial form on a display component, such as a CRT, LCD, LED, or OLED screen on a display device, or as a projection. However, other manners of displaying the icons may be devised. As a first alternative, the displaying may comprise printing a hard copy of the at least one icon of predominantly pictorial form, such that the icons representing the health condition of the individual are visible on a printed page or article. As a second alternative, the displaying may comprise sending the selected health condition descriptor and the selected health condition rating to a displaying component that is configured to display icons of predominantly pictorial form for the hierarchical classification scheme (e.g., by sending a video signal to a projector, or by sending the representation to a device that is configured to represent the descriptors of the health information hierarchical classification scheme via icons of a predominantly pictorial form.) Those of ordinary skill in the art may devise many ways of presenting the icons representing the selected health condition descriptor and the selected health condition rating in accordance with the techniques discussed herein.

In addition to the detecting, selecting, storing, and displaying features, other features may also be included in embodiments of these techniques that relate to the measured and represented aspect of the health state of the individual. As a first example, a diagnostic component may be included that includes with the detected measurements and/or representations a health diagnosis relating to the health condition. For instance, if the measurement comprises an intraocular pressure measurement, in addition to measuring the condition and storing the selected representation, these techniques may attempt to select and store a descriptor representing the health condition of the individual in view of the measurement. Accordingly, in one such embodiment, the hierarchical classification scheme comprises a set of health diagnoses, and the storing also comprising with the selected health condition descriptor and the selected health condition rating at least one health diagnosis related to the health condition. In some embodiments, the selecting of a diagnosis may be performed by a healthcare provider; accordingly, these techniques may include displaying the measurement for a healthcare provider of the individual, and upon receiving at least one health diagnosis from the healthcare provider related to the measurement, storing the at least one selected health diagnosis. In other embodiments, the diagnosis may be reached automatically by a diagnostic algorithm configured to compute the probabilities of various health diagnoses in view of the types of measurements captured by the detection component. One such diagnostic algorithm is disclosed in U.S. patent application Ser. No. 11/858,764 (“Automated Correlational Health Diagnosis”), the entirety of which (except the claims) is incorporated herein by reference.

Accordingly, in some embodiments of these techniques, the selecting may include selecting at least one health diagnosis related to the health condition according to the measurement; and the storing may comprise storing the at least one selected health diagnosis with the selected health condition descriptor and the selected health condition rating. Moreover, some embodiments may further represent and store the health treatments relating to the health diagnoses; e.g., the hierarchical classification scheme may include a set of health treatments relating to the health diagnoses, and the storing may comprise storing with the selected health condition descriptor, the selected health condition rating, and the at least one health diagnosis at least one health treatment related to at least one health diagnosis. By storing and representing the health diagnoses and/or treatments in relation to the health condition descriptors and health condition ratings, these techniques may be capable of displaying icons representing the diagnoses and treatments along with the representations of the measurements, thereby presenting a fuller representation of the electronic medical record and the health state of the individual to healthcare providers in the furtherance of healthcare. Those of ordinary skill in the art may be able to devise many ways for incorporating health diagnoses and/or treatments in the techniques discussed herein.

A second example of an additional feature that may be included in embodiments of these techniques relates to the communication of a detected measurement to a healthcare provider for the individual. A detecting performed in one location (such as a home healthcare setting, a medical lab, or an area of a hospital) may be sent to a healthcare provider, who may be located in a medical office or a different area of a hospital. The transmitted information may include one or more of the measurement, the selected health condition descriptor, and the selected health condition rating. The transmitted information may also include an annotation entered by the individual or another healthcare provider relating to the measured health condition. The transmission may also occur over many types of communications networks (e.g., a wired Ethernet network, a wireless network, or a cellular communication network), and according to many types of communications protocols (e.g., FTP, HTTP, or email.) The transmission may occur constantly (e.g., a continuing report of a heart monitor of a critically ill individual), and/or periodically (e.g., a weekly reporting of the blood pressure of an individual), and/or in response to a condition, such as an alert threshold that triggers an alerting of the healthcare provider in the case of a developing problem. Many techniques and motivations for communicating such health-related measurements and representations to healthcare providers may be devised by those of ordinary skill in the art while implementing the techniques discussed herein.

A third example of an additional feature that may be included in embodiments of these techniques relates to an administration of one or more medicaments, as may be directed by a healthcare provider or selected by an individual, in compliance with a medication regimen. For example, a device may be developed to facilitate a reminding or administration of an individual with regard to a medication regimen, such as the medication reminder device disclosed in U.S. patent application Ser. No. 11/712,376 (“Device for Facilitating Compliance With Medication Regimen”), and/or U.S. patent application Ser. No. 11/712,357 (“Method for Facilitating Compliance With Medication Regimen”), the entirety of which (except the claims) is incorporated by reference herein. It may be convenient to combine the detecting with the medication reminding and/or dispensing; e.g., an administration of a blood pressure medication may be coupled with a detecting of the blood pressure of the individual, which may further be reported to the healthcare provider for monitoring. Those of ordinary skill in the art may be able to devise many techniques for combining the detecting and monitoring features of the techniques discussed herein with other healthcare devices oriented for other healthcare tasks.

A second example of an additional feature that may be included in embodiments of these techniques relates to an administration of one or more medicaments, as may be directed by a healthcare provider or selected by an individual, in compliance with a medication regimen. For example, a device may be developed to facilitate a reminding or administration of an individual with regard to a medication regimen, such as the medication reminder device disclosed in U.S. patent application Ser. No. 11/712,376 (“Device for Facilitating Compliance With Medication Regimen”), the entirety of which (except the claims) is incorporated by reference herein. It may be convenient to combine the detecting with the medication reminding and/or dispensing; e.g., an administration of a blood pressure medication may be combined with a reminder to take blood pressure medication.

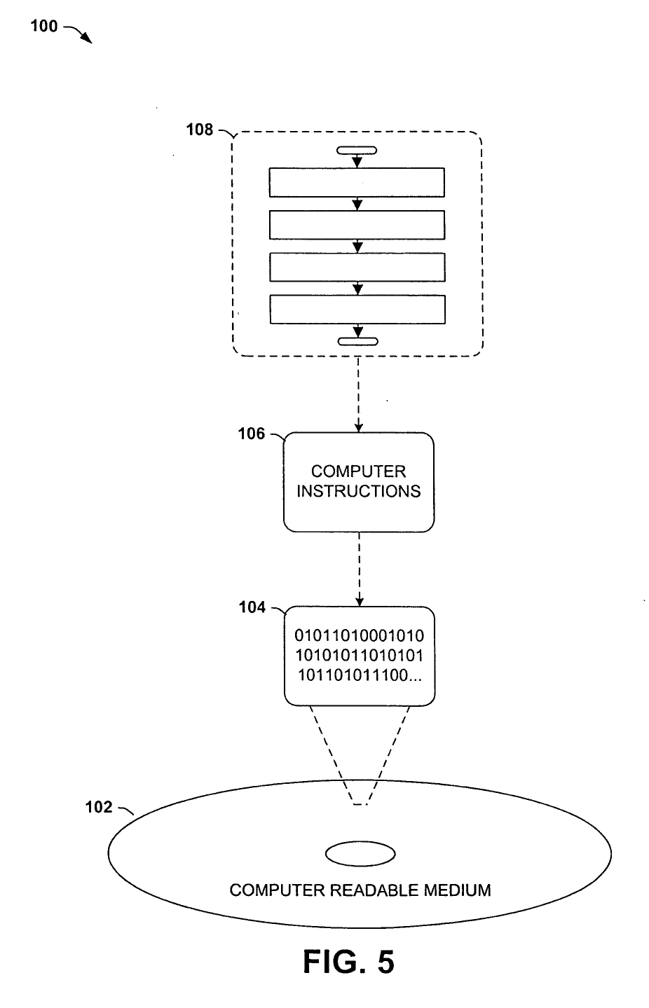

Still another embodiment involves a computer-readable medium comprising processor-executable instructions configured to apply the techniques presented herein. An exemplary computer-readable medium that may be devised in these ways is illustrated in FIG. 5, wherein the implementation 100 comprises a computer-readable medium 102 (e.g., a CD-R, DVD-R, or a platter of a hard disk drive), on which is encoded computer-readable data 104. This computer-readable data 104 in turn comprises a set of computer instructions 106 configured to operate according to the principles set forth herein. In one such embodiment, the processor-executable instructions 106 may be configured to perform a method of representing a health condition of an individual, such as the exemplary method 40 of FIG. 2. In another such embodiment, the processor-executable instructions 106 may be configured to implement a system for representing a health condition of an individual, such as the exemplary system 62 of FIG. 3. Many such computer-readable media may be devised by those of ordinary skill in the art that are configured to operate in accordance with the techniques presented herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used in this application, the terms “component,” “module,” “system”, “interface”, and the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, those skilled in the art will recognize many modifications may be made to this configuration without departing from the scope or spirit of the claimed subject matter.

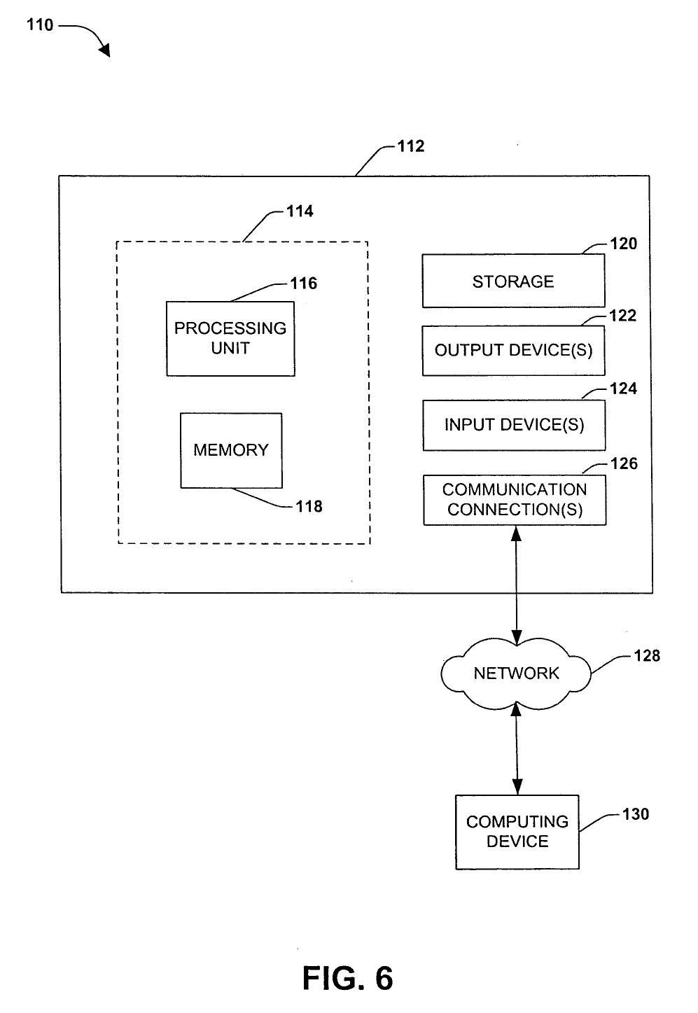

FIG. 6 and the following discussion provide a brief, general description of a suitable computing environment to implement embodiments of one or more of the provisions set forth herein. The operating environment of FIG. 6 is only one example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, Personal Digital Assistants (PDAs), media players, and the like), multiprocessor systems, consumer electronics, mini computers, mainframe computers, distributed computing environments that include any of the above systems or devices, and the like.

Although not required, embodiments are described in the general context of “computer readable instructions” being executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments.

FIG. 6 illustrates an example of a system 110 comprising a computing device 112 configured to implement one or more embodiments provided herein. In one configuration, computing device 112 includes at least one processing unit 116 and memory 118. Depending on the exact configuration and type of computing device, memory 118 may be volatile (such as RAM, for example), non-volatile (such as ROM, flash memory, etc., for example) or some combination of the two. This configuration is illustrated in FIG. 6 by dashed line 114.

In other embodiments, device 112 may include additional features and/or functionality. For example, device 112 may also include additional storage (e.g., removable and/or non-removable) including, but not limited to, magnetic storage, optical storage, and the like. Such additional storage is illustrated in FIG. 6 by storage 120. In one embodiment, computer readable instructions to implement one or more embodiments provided herein may be in storage 120. Storage 120 may also store other computer readable instructions to implement an operating system, an application program, and the like. Computer readable instructions may be loaded in memory 118 for execution by processing unit 116, for example.

The term “computer readable media” as used herein includes computer storage media. Computer storage media includes volatile and nonvolatile, removable and non-removable media implemented in any method or technology for storage of information such as computer readable instructions or other data. Memory 118 and storage 120 are examples of computer storage media. Computer storage media includes, but is not limited to, RAM, ROM, EEPROM, flash memory or other memory technology, CD-ROM, Digital Versatile Disks (DVDs) or other optical storage, magnetic cassettes, magnetic tape, magnetic disk storage or other magnetic storage devices, or any other medium which can be used to store the desired information and which can be accessed by device 112. Any such computer storage media may be part of device 112.

Device 112 may also include communication connection(s) 126 that allows device 112 to communicate with other devices. Communication connection(s) 126 may include, but is not limited to, a modem, a Network Interface Card (NIC), an integrated network interface, a radio frequency transmitter/receiver, an infrared port, a USB connection, or other interfaces for connecting computing device 112 to other computing devices. Communication connection(s) 126 may include a wired connection or a wireless connection. Communication connection(s) 126 may transmit and/or receive communication media.

The term “computer readable media” may include communication media. Communication media typically embodies computer readable instructions or other data in a “modulated data signal” such as a carrier wave or other transport mechanism and includes any information delivery media. The term “modulated data signal” may include a signal that has one or more of its characteristics set or changed in such a manner as to encode information in the signal.

Device 112 may include input device(s) 124 such as keyboard, mouse, pen, voice input device, touch input device, infrared cameras, video input devices, and/or any other input device. Output device(s) 122 such as one or more displays, speakers, printers, and/or any other output device may also be included in device 112. Input device(s) 124 and output device(s) 122 may be connected to device 112 via a wired connection, wireless connection, or any combination thereof. In one embodiment, an input device or an output device from another computing device may be used as input device(s) 124 or output device(s) 122 for computing device 112.

Components of computing device 112 may be connected by various interconnects, such as a bus. Such interconnects may include a Peripheral Component Interconnect (PCI), such as PCI Express, a Universal Serial Bus (USB), firewire (IEEE 1394), an optical bus structure, and the like. In another embodiment, components of computing device 112 may be interconnected by a network. For example, memory 118 may be comprised of multiple physical memory units located in different physical locations interconnected by a network.

Those skilled in the art will realize that storage devices utilized to store computer readable instructions may be distributed across a network. For example, a computing device 130 accessible via network 128 may store computer readable instructions to implement one or more embodiments provided herein. Computing device 112 may access computing device 130 and download a part or all of the computer readable instructions for execution. Alternatively, computing device 112 may download pieces of the computer readable instructions, as needed, or some instructions may be executed at computing device 112 and some at computing device 130.

Various operations of embodiments are provided herein. In one embodiment, one or more of the operations described may constitute computer readable instructions stored on one or more computer readable media, which if executed by a computing device, will cause the computing device to perform the operations described. The order in which some or all of the operations are described should not be construed as to imply that these operations are necessarily order dependent. Alternative ordering will be appreciated by one skilled in the art having the benefit of this description. Further, it will be understood that not all operations are necessarily present in each embodiment provided herein.

Moreover, the word “exemplary” is used herein to mean serving as an example, instance, or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims may generally be construed to mean “one or more” unless specified otherwise or clear from context to be directed to a singular form.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes”, “having”, “has”, “with”, or variants thereof are used in either the detailed description or the claims, such terms are intended to be inclusive in a manner similar to the term “comprising.”

What is claimed is:

1. A computer-implemented method of representing a health condition of an individual, the method comprising:
detecting a measurement of the health condition of the individual with a detection component;
selecting a health condition descriptor and a health condition rating that together describe the measurement from a health information hierarchical classification scheme having a set of health conditions, respective health conditions having a set of health condition descriptors and a set of health condition ratings;
storing the selected health condition descriptor and the selected health condition rating; and
displaying at least one icon of predominantly pictorial form that represents the selected health condition descriptor and the selected health condition rating.

2. The method of claim 1, the detection component comprising at least one of:
a visual detection component;
an audio detection component;
a temperature detection component;
a pressure detection component;
a flow detection component;
a chemical detection component;
a medical image detection component; and
a heart rhythm detection component.

3. The method of claim 1, the detection component comprising a user input component configured to receive input from a user comprising the health condition rating, the user comprising at least one of:
the individual, and
a healthcare provider of the individual.

4. The method of claim 3:
the detecting comprising: receiving from the user a health condition annotation through the user input component;
the storing comprising: storing the health condition annotation with the selected health condition descriptor and the selected health condition rating; and
the displaying comprising: displaying the health condition annotation with the at least one icon of predominantly pictorial form.

5. The method of claim 1, the selecting comprising: selecting a health condition rating for the health condition based on the measurement according to at least one of:
a qualitative measurement,
a quantitative measurement,
a measurement relative to a measurement standard,
a measurement relative to a prior measurement, and
a measurement relative to a predicted measurement.

6. The method of claim 1, the storing comprising: storing the measurement with the selected health condition descriptor and the selected health condition rating.

7. The method of claim 1:
the hierarchical classification scheme having a set of health diagnoses, and
the storing comprising: storing with the selected health condition descriptor and the selected health condition rating at least one health diagnosis related to the health condition.

8. The method of claim 7:
the selecting comprising: selecting at least one health diagnosis related to the health condition according to the measurement; and
the storing comprising: storing the at least one selected health diagnosis with the selected health condition descriptor and the selected health condition rating.

9. The method of claim 7, comprising:
displaying the measurement for a healthcare provider of the individual, and
upon receiving at least one health diagnosis from the healthcare provider related to the measurement, storing the at least one selected health diagnosis.

10. The method of claim 7:
the hierarchical classification scheme having a set of health treatments relating to the health diagnoses, and
the storing comprising: storing with the selected health condition descriptor, the selected health condition rating, and the at least one health diagnosis at least one health treatment related to at least one health diagnosis.

11. The method of claim 1, the displaying comprising: displaying the at least one icon of predominantly pictorial form by at least one of:
displaying the at least one icon of predominantly pictorial form on a display component;
printing a hard copy of the at least one icon of predominantly pictorial form; and
sending the selected health condition descriptor and the selected health condition rating to a displaying component configured to display icons of predominantly pictorial form for the hierarchical classification scheme.

12. The method of claim 1, comprising: communicating to a healthcare provider for the individual at least one of: the measurement, the selected health condition descriptor, and the selected health condition rating.

13. A computer-readable medium comprising processor-executable instructions configured to perform a computer-implemented method of representing a health condition of an individual, the method comprising:
detecting a measurement of the health condition of the individual with a detection component;
selecting a health condition descriptor and a health condition rating that together describe the measurement from a health information hierarchical classification scheme having a set of health conditions, respective health conditions having a set of health condition descriptors and a set of health condition ratings;
storing the selected health condition descriptor and the selected health condition rating; and
displaying at least one icon of predominantly pictorial form that represents the selected health condition descriptor and the selected health condition rating.

14. A system for representing a health condition of an individual, the system comprising:
a detecting component configured to detect a measurement of the health condition of the individual;
a health condition representing component configured to select a health condition descriptor and a health condition rating that together describe the measurement from a health information hierarchical classification scheme having a set of health conditions, respective health conditions having a set of health condition descriptors and a set of health condition ratings;
a health condition storing component configured to store the selected set of health conditions, respective health conditions having a set of health condition descriptors and a set of health condition ratings;
a displaying component configured to display at least one icon of predominantly pictorial form that represents the selected health condition descriptor and the selected health condition rating.

15. The system of claim 14, the detecting component comprising at least one of:
a visual detection component;
an audio detection component;
a temperature detection component;
a pressure detection component;
a flow detection component;
a chemical detection component;
a medical image detection component; and
a heart rhythm detection component.

16. The system of claim 14, comprising:
a user input component configured to receive input from a user comprising the health condition rating, the user comprising at least one of:
the individual, and
a healthcare provider of the individual.

17. The system of claim 14:
the hierarchical classification scheme having a set of health diagnoses; and
the health condition selecting component configured to select at least one health diagnosis related to the health condition according to the measurement; and
the health condition storing component configured to store with the selected health condition descriptor and the selected health condition rating the at least one selected health diagnosis.

18. The system of claim 17:
the hierarchical classification scheme having a set of health treatments relating to the health diagnoses;
the health condition selecting component configured to select at least one health treatment related to the at least one selected health diagnosis; and
the health condition storing component configured to store with the selected health condition descriptor, the selected health condition rating, and the at least one health diagnosis at least one health treatment related to at least one health diagnosis.

19. The system of claim 14, the displaying component comprising at least one of:
a visual displaying component configured to display the at least one icon of predominantly pictorial form; and
a printing component configured to print a hard copy of the at least one icon of predominantly pictorial form.

20. The system of claim 14, comprising:
a communications component configured to communicate to a healthcare provider for the individual at least one of: the measurement, the selected health condition descriptor, and the selected health condition rating.
